SEQUENCE LISTING (3 sequences)

SEQ ID NO: 1 - protein, 27 aa, Limulus polyphemus
HAEHKVKIKVKQKYGQFPQGTEVTYTC

SEQ ID NO: 2 - protein, 10 aa, Artificial Sequence
  Modified sequence from Homo sapiens
QRLFQVKGRR

SEQ ID NO: 3 - protein, 24 aa, Artificial Sequence
  Modified sequence from Lentivirus
RRWVRRVRRWVRRVVRVVRRWVRR

(19) United States
(12) Patent Application Publication
(10) Pub. No.: US 2024/0001017 A1
(43) Pub. Date: Jan. 4, 2024

(54) DEVICE FOR BINDING AND SEPARATION OF AT LEAST ONE COMPONENT FROM A BODY FLUID

(71) Applicant: Alteco Medical AB, Lund (SE)

(72) Inventor: Lennart Wilhelm Ljunggren, LUND (SE)

(21) Appl. No.: 18/039,419

(22) PCT Filed: Nov. 30, 2021
(86) PCT No.: PCT/EP2021/083593

§ 371 Date: May 30, 2023

(30) Foreign Application Priority Data
Nov. 30, 2020 (SE) 2051395-8

(51) Int. Cl. A61M 1/36; B01J 20/24; B01J 20/26; B01J 20/28

(52) U.S. Cl. CPC A61M 1/362; B01J 20/24; B01J 20/262; B01J 20/28004; B01J 20/28019; B01J 20/28038; B01J 20/28045; B01J 20/28052; A61M 1/3679

(57) ABSTRACT

A device for binding and separation of at least one component from a body fluid is disclosed. The device may have a proximal end and a distal end. The device may include a housing, an inlet disposed at the proximal end, an outlet, at least one of a sheet and a disc of a first matrix for binding of a first component from the body fluid, and a plurality of beads of a second matrix for binding of a second component from the body fluid. The first matrix may have a porous structure. The first component and the second component may be one of the same and different.

### CROSS-REFERENCE TO RELATED APPLICATIONS

This application claims priority to International Patent Application No. PCT/EP2021/083593, filed on Nov. 30, 2021, and Swedish Patent Application No. SE2051395-8, filed on Nov. 30, 2020, the contents of both of which are hereby incorporated by reference in their entirety.

### TECHNICAL FIELD

The present disclosure relates to a device for binding and separation of at least one component from a body fluid. More specifically, the present disclosure relates to a device for binding and separation of at least two components from a body fluid. The present disclosure also relates to a method for binding and separating at least one component from a body fluid. The present disclosure also relates to the use of a device according to the present disclosure.

### BACKGROUND

Inflammatory processes, such as inflammatory processes caused by infections or trauma, and autoinflammatory processes, such as inflammatory processes caused by immunological disorders, are a major cause of morbidity and mortality in humans.

It is estimated that, yearly, 400 000 to 500 000 episodes of sepsis result in 100 000 to 175 000 human deaths in the U.S. alone. In Germany, sepsis rates of up to 19% of patients stationed at intensive care units have been noted. Sepsis has also become the leading cause of death in intensive care units among patients with non-traumatic illnesses. Despite the major advances of the past decades in the treatment of serious infections, the incidence and mortality due to sepsis continues to rise.

There are three major types of sepsis characterized by the type of infecting organism. Gram-negative sepsis is the most common, caused by e.g. Escherichia coli. Gram-positive pathogens, such as the staphylococci and the streptococci, are the second major cause of sepsis.

A well-established mechanism in sepsis is related to a toxic component of Gram-negative bacteria, the lipopolysaccharide (LPS, endotoxin) cell wall structure, which is composed of a fatty acid group, a phosphate group, and a carbohydrate chain. Several of the host responses to LPS have been identified, such as release of cytokines, which are produced locally. In case of an extensive stimulation, however, there is a spill over to the peripheral blood and potential harmful effects are obtained, such as induced organ dysfunction.

Gram-positive bacteria produce the endotoxin lipoteichoic acid (LTA), which has similar effects as LPS.

Another substance released from Gram-positive bacteria is peptidoglycan, which also gives rise to an inflammatory response.

Several of the host responses to LPS have been identified, such as increased levels of lipopolysaccharide-binding protein (LBP), an acute phase protein, antimicrobial proteins such as heparin-binding protein (HBP), histones and high mobility group protein 1 (HMGB1), a pro-inflammatory protein. In addition, locally produced cytokines are released. HMBG1 causes overreaction of the inflammatory system. HBP, LBP and histones contribute to endothelial dysfunction.

The key mediators of septic shock are Tumor Necrosis Factor (TNF-α), Interleukine 1 (II-1) and Interleukine 17 (II-17), which are released by monocytes and macrophages. They act synergistically causing a cascade of physiological changes leading to circulation collapse and multi organ failure. Other cytokines involved in inflammatory processes are IL-6, IL-8, IL-12, IL-18, and IFN-γ.

Antibiotics of varying types are widely used to prevent and treat infections. However, for many commonly used antibiotics an antibiotic resistance has developed among various species of bacteria. Antibiotics can be toxic to varying degrees by causing allergy, interactions with other drugs, and causing direct damage to major organs (e.g. liver and kidney). Many antibiotics also change the normal intestinal flora, which can cause diarrhea and nutritional malabsorption.

Endogenous substances that may elicit an inflammatory response include the complement factors C3a, C35a, histones and autoantibodies. These substances may cause the release of inflammatory cytokines, e.g. IL-1, IL-6, IL-8, IL-12, IL-17, IL-18, IFN-γ and TNF-α.

In attempts to remove components from blood, different adsorbent materials have been prepared. An endotoxin removal adsorbent comprising a ligand immobilized on a solid phase support medium is shown in WO 01/23413. A preferred support medium is in the form of beads. When packed in a separation device, the solid phase support medium is porous enough to allow passage of blood cells between the beads.

EP 1 497 025 B1 discloses a method for selectively binding and separating at least one component from a body fluid without the need of separating blood into plasma and blood cells. The component may be LPS. The body fluid is passed through a rigid integral separation matrix whereby the component binds to at least one functional group in the matrix.

The simultaneous removal of more than one component from blood is described in e.g. EP 3 600 485, US 20020146413.

An object of the present disclosure is to provide an improved device for selective binding and separating at least one component from whole blood or body fluids.

### SUMMARY

According to a first aspect, the above and other objects of the disclosure are achieved, in full or at least in part, by a device as defined by claim 1. According to this claim the above and other objects are achieved by a device for binding and separation of at least one component from a body fluid, the device having a proximal end and a distal end and comprising a housing; an inlet located at the proximal end; an outlet; at least one sheet or disc of a first matrix for binding of a first component from a body fluid, wherein the first matrix has a porous structure; and beads of a second matrix (6) for binding of a second component from a body fluid; wherein the first component from a body fluid and the second component from a body fluid are the same or different.

According to a second aspect, the above and other objects are achieved by a method for binding and separating at least one component from a body fluid, comprising the step of: introducing a body fluid through the inlet of the device according to the present disclosure; passing the body fluid through the first matrix and the second matrix, and, if present, through further present matrices, of the device according to the present disclosure, whereby said at least one component binds to at least one of the matrices; and collecting the body fluid from the outlet of the device according to the present disclosure.

According to a second aspect, the above and other objects are achieved by a use of a device according to the present disclosure for binding and separation of at least one component from a body fluid.

Other objectives, features and advantages of the present disclosure will appear from the following detailed description, from the drawings, as well as from the attached claims. It is noted that the disclosure relates to all possible combination of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of said element, device, component, means, step, etc., unless explicitly stated otherwise. The steps of any method disclosed herein do not have to be performed in the exact order disclosed, unless explicitly stated.

As used herein, the term “comprising” and variations of that term are not intended to exclude other additives, components, integers or steps.

### BRIEF DESCRIPTION OF THE DRAWINGS

By way of example, embodiments of the present disclosure will now be described with reference to the accompanying drawings, in which:

FIGS. 1a-1e are schematic drawings of devices according to the present disclosure.

FIGS. 2a-2b are schematic drawings of a device according to the present disclosure.

FIG. 3a-3b are schematic drawings of devices according to the present disclosure.

FIGS. 4a-4d are schematic drawings of cross sections of devices according to the present disclosure.

### DETAILED DESCRIPTION

The present disclosure relates to devices for binding and separation of at least one component from a body fluid. The present disclosure also relates to a method for binding and separating at least one component from a body fluid. The present disclosure also relates to the use of a device according to the present disclosure binding and separation of at least one component from a body fluid.

Specific aspects and embodiments of the present disclosure will be described in detail below.

Device

The devices disclosed herein are for binding and separation of at least one component from a body fluid.

General features applicable to devices disclosed herein as well as specific examples of such devices are described below.

A device 1 according to the present disclosure has a proximal end A and a distal end B and comprises a housing 2; an inlet 3 located at the proximal end A; an outlet 4; at least one sheet or disc of a first matrix 5 for binding of a first component from a body fluid, wherein the first matrix has a porous structure; and beads of a second matrix 6 for binding of a second component from a body fluid; wherein the first component from a body fluid and the second component from a body fluid are the same or different.

The sheets or discs of the first matrix 5 prevent channeling and distributes the body fluid over the area of the sheet or discs, thus creating a substantially uniform distribution of the body fluid. Thus, the sheets or discs of the first matrix 5 distribute the flow of the body fluid over substantially the entire area of the cross section of the device, such that more of the available adsorption capacity of the beads of the second matrix 6 can be utilized. Further, channeling in the beads is reduced. Thus, less beads may be used to achieve the same adsorption capacity of a device not comprising the discs. In addition, when passing through the sheets or discs, the body fluid is mixed. This leads to even concentrations of the components on the body fluid. Thus, concentration gradients are avoided and an increased part of the body fluid is in contact with the matrices.

The beads of the second matrix 6 and of the fourth matrix 8 provide a large surface are where a component of the body fluid can bind.

The binding may be such that the component that is bound to a matrix may be eluted from the device. In such cases, the device can be used again.

The binding may be selective binding of a specific component.

The binding may be due to adsorption, hydrophobic interactions, hydrophilic interactions and ionic interactions. Thus, a component may be adsorbed to a matrix.

The body fluid may be whole blood, plasma or cerebrospinal fluid.

Preferably, the body fluid is whole blood.

The outlet may be located at the proximal end A of the device.

Alternatively, the outlet 4 is located on the sidewall of the device.

Preferably, the outlet 4 is located at the distal end B of the device.

One of the first component and the second component, which are to be bound and separated, may be an exogenous component, i.e. a component that is not produced by the patient whose body fluid, such as whole blood, is to be passed through the device.

Examples of such components may be a toxic component produced by infectious agent, such as from a bacterium, a virus or a fungus.

More specifically, the component may be derived from a bacterium.

One of the first component and the second component, which are to be bound and separated, may be an endogenous component, i.e. a component produced by the patient whose body fluid, such as whole blood, is to be passed through the device.

The first component and/or the second component may be chosen from the group consisting of: an endotoxin, preferably wherein the endotoxin is LPS, LTA and/or peptidoglycan; a cytokine, preferably wherein the cytokine is a pro-inflammatory cytokine or an anti-inflammatory cytokine, more preferably wherein the cytokine is chosen from the group consisting of IL-1, IL-6, IL-8, IL-12, IL-17, IL-18, IFN-γ and TNF-α; a complement factor, preferably wherein the complement factor is C3a or C5a; histone; an autoantibody; macrophage migration inhibitory factor (MIF); high mobility group protein 1 (HMGB1); heparin-binding protein (HBP); lipopolysaccharide-binding protein (LBP); DNA; and fragments thereof.

A preferred example of a component, which is to be bound and separated in a device disclosed herein, is lipopolysaccharide (LPS), or a fragment thereof, produced by Gram-negative bacteria or lipoteichoic acid (LTA), or a fragment thereof, produced by Gram-positive bacteria or peptidoglycan produced by bacteria.

Thus, preferred examples of the device relate to the selective binding of LPS, or a fragment thereof, and to the separation of toxic LPS, or a fragment thereof, from the body fluid, such as from the whole blood, of a patient suffering from sepsis caused by Gram-negative bacteria.

Other preferred examples of the device relate to the selective binding of LTA, or a fragment thereof, and to the separation of toxic LTA, or a fragment thereof, from the body fluid, such as from the whole blood, of a patient suffering from sepsis caused by Gram-positive bacteria.

Other preferred examples of the device relate to the selective binding of peptidoglycan, or a fragment thereof, and to the separation of peptidoglycan, or a fragment thereof, from the body fluid, such as from the whole blood, of a patient suffering from sepsis caused by bacteria.

Another preferred example of a component, which is to be bound and separated in a device disclosed herein, is a pro-inflammatory cytokine, such as e.g. IL-1, IL-6, IL-8, IL-12, IL-17, IL-18, IFN-γ or TNF-α, or fragments thereof.

Another preferred example of a component, which is to be bound and separated in a device disclosed herein is IL-1, or a fragment thereof.

Another preferred example of a component, which is to be bound and separated in a device disclosed herein is IL-6, or a fragment thereof.

Another preferred example of a component, which is to be bound and separated in a device disclosed herein is IL-8, or a fragment thereof.

Another preferred example of a component, which is to be bound and separated in a device disclosed herein is IL-12, or a fragment thereof.

Another preferred example of a component, which is to be bound and separated in a device disclosed herein is IL-17, or a fragment thereof.

Another preferred example of a component, which is to be bound and separated in a device disclosed herein is IL-18, or a fragment thereof.

Another preferred example of a component, which is to be bound and separated in a device disclosed herein is IFN-γ, or a fragment thereof.

Another preferred example of a component, which is to be bound and separated in a device disclosed herein is TNF-α, or a fragment thereof.

Another preferred example of a component, which is to be bound and separated in a device disclosed herein, is a complement factor, such as e.g. C3a or C5a, or fragments thereof.

Another preferred example of a component, which is to be bound and separated in a device disclosed herein, is histone, or a fragment thereof. Preferably, the histone is an extracellular histone.

Another preferred example of a component, which is to be bound and separated in a device disclosed herein, is an autoantibody, or a fragment thereof.

Another preferred example of a component, which is to be bound and separated in a device disclosed herein, is macrophage migration inhibitory factor (MIF), or a fragment thereof.

Another preferred example of a component, which is to be bound and separated in a device disclosed herein, is high mobility group protein 1 (HMGB1), or a fragment thereof.

Another preferred example of a component, which is to be bound and separated in a device disclosed herein, is heparin-binding protein (HBP), or a fragment thereof.

Another preferred example of a component, which is to be bound and separated in a device disclosed herein, is lipopolysaccharide-binding protein (LBP), or a fragment thereof.

Another preferred example of a component, which is to be bound and separated in a device disclosed herein, is DNA, preferably mitochondrial DNA.

According to another embodiment, the first component is LPS or LTA and the second component is a pro-inflammatory cytokine or an anti-inflammatory cytokine, preferably a pro-inflammatory cytokine.

The pro-inflammatory cytokine may be IL-1, IL-6, IL-8, IL-12, IL-17, IL-18, IFN-γ or TNF-α, or fragments thereof.

According to yet another embodiment, the first matrix 5 comprises a moiety that specifically binds the first component. Preferably, said moiety is chosen from the group consisting of a peptide; a peptoid; an antibody; an antibody fragment; a receptor; a receptor fragment; an aptamer; an aptamer fragment; a polysaccharide; and a polysaccharide fragment.

LPS may be bound by a peptide. Preferably, the LPS-binding peptide is 2 to 40 amino acids long, more preferably 4 to 35 amino acids long, and in some cases 20 to 30 amino acids long.

LPS may be bound by a peptide according to SEQ ID NO 1 or a peptide having at least 80%, 85%, 90%, 95% or 99% homology with the peptide according to SEQ ID NO 1.

Alternatively, LPS may be bound by a peptide according to SEQ ID NO 2 or a peptide having at least 80%, 85%, 90%, 95% or 99% homology with the peptide according to SEQ ID NO 2.

Alternatively, LPS may be bound by a peptide according to SEQ ID NO 3 or a peptide having at least 80%, 85%, 90%, 95% or 99% homology with the peptide according to SEQ ID NO 3.

LPS may be bound by an LPS-specific aptamer or fragment thereof.

LTA may be bound by a peptide according to SEQ ID NO 2 or a peptide having at least 80%, 85%, 90%, 95% or 99% homology with the peptide according to SEQ ID NO 2.

Alternatively, LTA may be bound by a peptide according to SEQ ID NO 3 or a peptide having at least 80%, 85%, 90%, 95% or 99% homology with the peptide according to SEQ ID NO 3.

LTA may be bound by an LTA-specific aptamer or fragment thereof.

Peptidoglycan may be bound by specific antibodies or antibody fragments or peptidoglycan-binding peptides, such as melittin.

Cytokines may be bound by specific antibodies or antibody fragments.

Alternatively, cytokines may be bound by specific receptors or receptor fragments.

Complement factors may be bound by specific antibodies or antibody fragments.

Alternatively, complement factors may be bound by specific receptors or receptor fragments.

Histone may be bound by specific antibodies or antibody fragments.

An autoantibody may be bound by specific antigens.

Macrophage migration inhibitory factor (MIF) may be bound by specific antibodies or antibody fragments. Alternatively, MIF may be bound by specific receptors or receptor fragments.

High mobility group protein 1 (HMGB1) may be bound by specific antibodies or antibody fragments. Alternatively, HMGB1 may be bound by specific receptors or receptor fragments. HMGB1 may be bound by heparin (a polysaccharide) or fragments thereof.

Heparin-binding protein (HBP) may be bound by specific antibodies or antibody fragments. Alternatively, HBP may be bound by specific receptors or receptor fragments. HBP may be bound by heparin (a polysaccharide) or fragments thereof.

Lipopolysaccharide-binding protein (LBP) may be bound by specific antibodies or antibody fragments. Alternatively, LBP may be bound by specific receptors or receptor fragments. LBP may be bound by heparin (a polysaccharide) or fragments thereof.

DNA may be bound by specific aptamers or aptamer fragments.

As stated above, the first component and the second component may be the same. This is especially advantageous since the component-binding surface area is increased in a device according to the present disclosure in relation to a device of the same volume, but having only rigid matrix. In relation to a device having only beads, a device according to the present disclosure provides better mixing of the body fluid, prevents channeling and provides an even distribution of the body fluid.

As stated above, the first component and the second component may be different. For example, the first component may be LPS or LTA and the second component may be a pro-inflammatory cytokine, such as IL-1, IL-6, IL-8, IL-12, IL-17, IL-18, IFN-γ or TNF-α. An advantage of this arrangement is that both the cause (LPS or LTA) of the inflammatory response as well as the endogenous mediators of the inflammations (pro-inflammatory cytokines) are bound by the matrices of the device 1 and are separated from the blood or plasma which is passed through the device 1.

The First Matrix 5

According to one embodiment, the first matrix comprises an endotoxin-binding peptide, wherein the endotoxin-binding peptide is chosen from the group consisting of an LPS-binding peptide according to SEQ ID NO 1; a peptide having at least 80%, 85%, 90%, 95% or 99% homology with the LPS-binding peptide according to SEQ ID NO 1; an LPS/LTA-binding peptide according to SEQ ID NO 2; and a peptide having at least 80%, 85%, 90%, 95% or 99% homology with the LPS/LTA-binding peptide according to SEQ ID NO 2; an LPS/LTA-binding peptide according to SEQ ID NO 3; and a peptide having at least 80%, 85%, 90%, 95% or 99% homology with the LPS/LTA-binding peptide according to SEQ ID NO 3.

The first matrix 5 may be in the form of a sheet, having a thickness of 1 to 10 mm, such as 2 to 8 mm, such as 4 to 6 mm, preferably about 5 mm.

Preferably, the first matrix 5 is in the form of a disc, having a thickness of 1 to mm, such as 2 to 8 mm, such as 4 to 6 mm, preferably about 5 mm. The diameter of such a disc may be 2 to 15 cm, such as 3 to 10 cm, such as 4 to 8 cm, preferably around cm.

The first matrix 5 has a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The first matrix 5 preferably has an active surface ranging from 0.5 cm² to 10 m², preferably 4 cm² to 6 m², as measured by the BET-method, measured either by nitrogen adsorption or mercury intrusion.

According to another embodiment, the first matrix 5 is chosen from the group consisting of a foamed polymer, a molded polymer, a sintered polymer, a polymer cryogel, a nonwoven fabric, and a molecular imprinted polymer that binds the first component.

Preferably, the first matrix 5 is a sintered polymer. The polymer may be a polyolefine, such as polyethylene, polypropylene, polybutylene, polymetylpentene, and ethylene vinyl acetate copolymers; vinylic polymers, such as polyvinyl alcohol, polyvinyl acetals, and polyvinylpyrrolidone; polyacrylates, such as poly-methylmethacrylate, cyanoacrylate, polyacrylonitrile, and polymetacrylates; polyamides, such as polyacrylamide; polyimides, such as polyethylenimines; polystyrene and its copolymers, such as polystyrene and acrylonitrile-butadiene-styrene-polymers; silicone rubbers; polyesters/ethers; poly carbonates; polyurethanes; polysulfonates; polygly cols; polyalkydeoxides such as poly ethyl eneoxide, polypropyleneoxide; and copolymers or hybrids or mixtures thereof.

Other examples of suitable synthetic polymers are cyclic olefins and copolymers thereof.

Preferably, the first matrix 5 is made from synthetic polymers, more preferably polyolefins, such as polyethylene or polypropylene or mixtures thereof.

Especially, sintered synthetic polymers, such as sintered polyolefins, such as polyethylene or polypropylene or mixtures thereof, are preferred.

Thus, according to another embodiment, the first matrix 5 is sintered polyethylene. The sintered polyethylene has a porous structure. Preferably, the pore size ranges from 1 μm to 500 μm in diameter, more preferably from 70 μm to 170 μm, most preferred from 80 μm to 100 When the body fluid does not contain any blood cells, the pore size may be 1 μm to 25 μm. Preferably, the active surface of a matrix according to the present disclosure ranges from 0.5 cm² to 10 m², preferably 4 cm² to 6 m², as measured by the BET-method, measured either by nitrogen adsorption or mercury intrusion.

Alternatively, the first matrix 5 is a nonwoven fabric. A nonwoven fabric is a material of fibers or filaments that have not been converted into yarns, but are bonded together. The nonwoven fabric may be made from natural or synthetic polymers. The shape of the fibers used may be e.g. cylindrical, star or T structures. The nonwoven fabric may be wet-bonded, dry-bonded or spun-bonded. The diameter of the fibers used may vary from nm to mm. Nonwoven fabrics made from fibers with smaller diameter have a larger surface area than nonwoven fabrics made from fibers with a larger diameter. Moreover, different nonwoven fabrics may be used separately or in combinations.

The first matrix 5 may comprise a peptide that binds the first component.

According to one alternative, the peptide may be covalently attached to polydopamine coupled to the first matrix. The peptide may be coupled to the polydopamine via an amine-group or via a thiol-group.

According to another alternative, the peptide may be covalently attached to the matrix via a linker covalently bound to a residue of an amino-group present in the matrix. The amino-group may be part of the material used to produce the matrix or may be added by functionalization of the material used to produce the matrix. Amino-groups may be introduced by coupling polydopamine bound to an amine, an oligoamine or a polyamine, to the first matrix 5. In such a case, the linker is covalently bound to the amine, oligoamine or polyamine, which is attached to the first matrix 5 via polydopamine.

The linker may be a homobifunctional cross-linker or a heterobifunctional cross-linker. When the linker is a homobifunctional cross-linker, the linker is covalently bound to a residue of an amino-group in the matrix and to an amino-group present in the peptide. Examples of such homobifunctional cross-linkers include glutardialdehyde and diepoxides such as poly(ethylene glycol) diglycidyl ether and 1-4-butanediol diglycidyl ether.

Preferably, the linker is a heterobifunctional cross-linker. Such linkers bind to two different functional groups. When the linker is a heterobifunctional cross-linker, the linker is covalently bound to a residue of an amino-group in the matrix and to a thiol-group present in the peptide. By binding to two different functional groups, the risk of the improper binding of the peptide is reduced.

Preferably, the linker is a heterobifunctional cross-linker chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether. Such linkers have been shown to bind the peptide in a stable manner to the matrix. Since the linker binds to two different functional groups, the risk of the improper binding of the peptide is reduced.

Preferably, the linker is SMCC, CBTF, maleimide-poly(ethylene glycol)-succinimidyl ester, poly(ethylene glycol)-diglycidyl ether or 1-4-butanediol diglycidyl ether.

Most preferably, the linker is SMCC or CBTF.

The linkers disclosed herein act as coupling agents, coupling the peptide to the matrix. In addition, the linkers also provide a means to create a distance between the matrix and the peptide, such that the peptide is presented to the component that is to be bound by the matrix. If the peptide is too close to the surface of the matrix, possible available sites of interaction with the component are limited. Thus, by creating a distance between the matrix and the peptide, the availability of the peptide to the components present in the body fluid is increased, thereby increasing the number of binding sites for the component in the matrix. Preferably, the linker creates a distance from the surface of the matrix to the peptide of 6 carbon atoms or more.

When the first matrix 5 comprises a peptide, the matrix may be coated with a polyalcohol. Such a coating stabilizes the peptide. This is especially advantageous when the matrix is stored under dry conditions. The polyalcohol acts as a humectant, thus stabilizing the peptide. Moreover, the polyalcohol may act as a bacteriostatic compound, preventing the growth of bacteria in the matrix during the production before the matrix is sterilized. In other words, the polyalcohol prevents an increased bioburden. Furthermore, if the matrix is made of a partly hydrophobic material, such as e.g. polyethylene, hydrophobic parts of the peptide may interact with hydrophobic parts of the matrix material and thus loose its ability to bind to the component, which is to be bound by the matrix. By coating the matrix, i.e. providing a conjugated matrix, with a polyalcohol, hydrophobic parts of the matrix material are made more hydrophilic, since hydrophobic parts of the polyalcohol interact with the hydrophobic parts of the matrix material and the hydrophilic parts of the polyalcohol are faced towards the peptide, thereby hindering the hydrophobic interaction between the peptide and the matrix material.

Before the matrix is used, it is rinsed with a physiological NaCl-solution to remove the polyalcohol. It is advantageous if the polyalcohol is biocompatible, i.e. non-toxic, in the event smaller residual amounts of the polyalcohol are left after rinsing.

Examples of biocompatible polyalcohols, which may be used, are propane-1,2,3-triol, glucose, trehalose and a mixture thereof. Preferably, the polyalcohol is propane-1,2,3-triol, also known as glycerol or glycerin(e). Propane-1,2,3-triol is an endogenous non-toxic compound, making it especially suitable for coating of the matrix. Furthermore, it is a liquid at room temperature and is thus easy to handle. Further, since it is a liquid it will not evaporate or crystallize and is thus an especially effective humectant.

Another advantage of the presence of a polyalcohol-coating is that such a coating acts as a radical-scavenger, thereby protecting the matrix from beta- and gamma-radiation, which may be used for sterilizing the matrix.

Preferably, the polyalcohol is used in an amount corresponding to up to 0.1 to g/m² of the matrix.

The Second Matrix 6

According to one embodiment, the second matrix 6 comprises a moiety that specifically binds the second component. Preferably, said moiety is chosen from the group consisting of a peptide; a peptoid; an antibody; an antibody fragment; a receptor; a receptor fragment; an aptamer; an aptamer fragment; a polysaccharide; and a polysaccharide fragment; and/or preferably wherein said second component is a pro-inflammatory cytokine, an anti-inflammatory cytokine or a complement factor.

LPS may be bound by a peptide. Preferably, the LPS-binding peptide is 4 to 40 amino acids long, more preferably 10 to 35 amino acids long, and in some cases 20 to amino acids long.

LPS may be bound by a peptide according to SEQ ID NO 1 or a peptide having at least 80%, 85%, 90%, 95% or 99% homology with the peptide according to SEQ ID NO 1.

Alternatively, LPS may be bound by a peptide according to SEQ ID NO 2 or a peptide having at least 80%, 85%, 90%, 95% or 99% homology with the peptide according to SEQ ID NO 2.

Alternatively, LPS may be bound by a peptide according to SEQ ID NO 3 or a peptide having at least 80%, 85%, 90%, 95% or 99% homology with the peptide according to SEQ ID NO 3.

LPS may be bound by an LPS-specific aptamer or fragment thereof.

LTA may be bound by a peptide according to SEQ ID NO 2 or a peptide having at least 80%, 85%, 90%, 95% or 99% homology with the peptide according to SEQ ID NO 2.

Alternatively, LTA may be bound by a peptide according to SEQ ID NO 3 or a peptide having at least 80%, 85%, 90%, 95% or 99% homology with the peptide according to SEQ ID NO 3.

LTA may be bound by an LTA-specific aptamer or fragment thereof.

Peptidoglycan may be bound by specific antibodies or antibody fragments or peptidoglycan-binding peptides, such as melittin.

Cytokines may be bound by specific antibodies or antibody fragments.

Alternatively, cytokines may be bound by specific receptors or receptor fragments.

Complement factors may be bound by specific antibodies or antibody fragments.

Alternatively, complement factors may be bound by specific receptors or receptor fragments.

Histone may be bound by specific antibodies or antibody fragments.

An autoantibody may be bound by specific antigens.

Macrophage migration inhibitory factor (MIF) may be bound by specific antibodies or antibody fragments. Alternatively, MIF may be bound by specific receptors or receptor fragments.

High mobility group protein 1 (HMGB1) may be bound by specific antibodies or antibody fragments. Alternatively, HMGB1 may be bound by specific receptors or receptor fragments. HMGB1 may be bound by heparin (a polysaccharide) or fragments thereof.

Heparin-binding protein (HBP) may be bound by specific antibodies or antibody fragments. Alternatively, HBP may be bound by specific receptors or receptor fragments. HBP may be bound by heparin (a polysaccharide) or fragments thereof.

Lipopolysaccharide-binding protein (LBP) may be bound by specific antibodies or antibody fragments. Alternatively, LBP may be bound by specific receptors or receptor fragments. LBP may be bound by heparin (a polysaccharide) or fragments thereof.

DNA may be bound by specific aptamers or aptamer fragments.

According to another embodiment, the beads of the second matrix 6 are beads of agarose or beads of polyacrylate, polystyrene, polystyrenedivinylbenzene or copolymers thereof.

According to yet another embodiment, the beads of the second matrix 6 have a diameter of 20 to 1,500 μm and/or a pore size of 10 to 1,000 nm.

When the body fluid is whole blood, the diameter of the beads is preferably 150 to 1,500 μm, such as 200 to 1,000 μm, such as 220 to 750 μm. such as 240 to 500 μm, most preferred 250 to 350 μm.

When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm.

The beads of the second matrix 6 may have a porous structure. The pore size preferably ranges from 10 nm to 1,000 nm.

In some examples, the pore size is preferably 10 to 300 nm, such as 50 to 200 nm, such as 75 to 150 nm, such as 100 nm.

In some examples, the pore size is preferably 300 nm to 1,000 nm, such as 400 to 750 nm, such as 450 to 600 nm, such as 500 nm.

Embodiments of the Device Comprising More than Two Matrices

According to one embodiment, the device 1 comprises at least two sheets or discs of the first matrix 5.

According to another embodiment, the at least two sheets or discs of the first matrix (5) are separated from each other by at least one layer comprising the beads of the second matrix (6).

According to yet another embodiment, the device 1 further comprises: at least one sheet or disc of a third matrix 7 for binding of a third component from a body fluid, wherein the third matrix has a porous structure; and/or beads of a fourth matrix (8) for binding of a fourth component from a body fluid.

According to a further embodiment, the third and/or the fourth component is chosen from the group consisting of: an endotoxin, preferably wherein the endotoxin is LPS, LTA and/or peptidoglycan; a cytokine, preferably wherein the cytokine is a pro-inflammatory cytokine or an anti-inflammatory cytokine, more preferably wherein the cytokine is chosen from the group consisting of IL-1, IL-6, IL-8, IL-12, IL-17, IL-18, IFN-γ and TNF-α; a complement factor, preferably wherein the complement factor is C3a or C5a; histone; an autoantibody; macrophage migration inhibitory factor (MIF); high mobility group protein 1 (HMGB1); heparin-binding protein (HBP); lipopolysaccharide-binding protein (LBP); DNA; and fragments thereof.

Thus, the third component is chosen from the group consisting of: an endotoxin, preferably wherein the endotoxin is LPS, LTA and/or peptidoglycan; a cytokine, preferably wherein the cytokine is a pro-inflammatory cytokine or an anti-inflammatory cytokine, more preferably wherein the cytokine is chosen from the group consisting of IL-1, IL-6, IL-8, IL-12, IL-17, IL-18, IFN-γ and TNF-α; a complement factor, preferably wherein the complement factor is C3a or C5a; histone; an autoantibody; macrophage migration inhibitory factor (MIF); high mobility group protein 1 (HMGB1); heparin-binding protein (HBP); lipopolysaccharide-binding protein (LBP); DNA; and fragments thereof.

Thus, the fourth component is chosen from the group consisting of: an endotoxin, preferably wherein the endotoxin is LPS, LTA and/or peptidoglycan; a cytokine, preferably wherein the cytokine is a pro-inflammatory cytokine or an anti-inflammatory cytokine, more preferably wherein the cytokine is chosen from the group consisting of IL-1, IL-6, IL-8, IL-12, IL-17, IL-18, IFN-γ and TNF-α; a complement factor, preferably wherein the complement factor is C3a or C5a; histone; an autoantibody; macrophage migration inhibitory factor (MIF); high mobility group protein 1 (HMGB1); heparin-binding protein (HBP); lipopolysaccharide-binding protein (LBP); DNA; and fragments thereof.

The third and fourth component may be the same or different.

According to another embodiment, the third matrix 7 comprises a moiety that specifically binds the third component and/or the fourth matrix 8 comprises a moiety that specifically binds the fourth component. Preferably, said moieties are independently chosen from the group consisting of a peptide; an antibody; an antibody fragment; a receptor; a receptor fragment; an aptamer; an aptamer fragment; a polysaccharide; and a polysaccharide fragment.

Different components of the body fluid may be bound by different moieties as described above in relation to the first matrix 5 and the second matrix 6.

Thus, the third matrix 7 may comprise a moiety that specifically binds the third component. Preferably, the moiety is chosen from the group consisting of a peptide; an antibody; an antibody fragment; a receptor; a receptor fragment; an aptamer; an aptamer fragment; a polysaccharide; and a polysaccharide fragment.

Thus, the fourth matrix 8 may comprise a moiety that specifically binds the fourth component. Preferably, the moiety is chosen from the group consisting of a peptide; an antibody; an antibody fragment; a receptor; a receptor fragment; an aptamer; an aptamer fragment; a polysaccharide; and a polysaccharide fragment.

The moiety of the third matrix 7 and the moiety of the fourth matrix 8 are preferably independently chosen from the group consisting of a peptide; an antibody; an antibody fragment; a receptor; a receptor fragment; an aptamer; an aptamer fragment; a polysaccharide; and a polysaccharide fragment.

The Third Matrix

The third matrix 7 may comprise an endotoxin-binding peptide, wherein the endotoxin-binding peptide is chosen from the group consisting of an LPS-binding peptide according to SEQ ID NO 1; a peptide having at least 80%, 85%, 90%, 95% or 99% homology with the LPS-binding peptide according to SEQ ID NO 1.

The third matrix 7 may comprise an LPS/LTA-binding peptide according to SEQ ID NO 2; and a peptide having at least 80%, 85%, 90%, 95% or 99% homology with the LPS/LTA-binding peptide according to SEQ ID NO 2.

The third matrix 7 may comprise an LPS/LTA-binding peptide according to SEQ ID NO 3; and a peptide having at least 80%, 85%, 90%, 95% or 99% homology with the LPS/LTA-binding peptide according to SEQ ID NO 3.

The third matrix 7 may be in the form of a sheet, having a thickness of 1 to 10 mm, such as 2 to 8 mm, such as 4 to 6 mm, preferably about 5 mm.

Preferably, the third matrix 7 is in the form of a disc, having a thickness of 1 to mm, such as 2 to 8 mm, such as 4 to 6 mm, preferably about 5 mm. The diameter of such a disc may be 2 to 15 cm, such as 3 to 10 cm, such as 4 to 8 cm, preferably around cm.

The third matrix 7 has a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The third matrix 7 may have an active surface as described above for the first matrix 5.

The third matrix 7 may be chosen from the group consisting of a foamed polymer, a molded polymer, a sintered polymer, a polymer cryogel, a nonwoven fabric, and a molecular imprinted polymer that binds the first component.

Preferably, the third matrix 7 is a sintered polymer. The polymer may be as described above for the first matrix 5.

Preferably, the third matrix 7 is sintered polyethylene. The sintered polyethylene may be functionalized with amino-groups as detailed above.

The third matrix 7 may comprise a peptide that binds the third component. The peptide may be covalently attached to the matrix via a linker covalently bound to a residue of an amino-group present in the matrix as detailed above in relation to the first matrix 5.

The third matrix 7 may be coated with a polyalcohol as detailed above in relation to the first matrix 5.

The Fourth Matrix

The beads of the fourth matrix 8 may be beads of agarose or beads of polyacrylate, polystyrene, polystyrenedivinylbenzene or copolymers thereof.

Preferably, the beads of the fourth matrix 8 have a diameter of 20 to 1,500 μm and/or a pore size of 10 to 1,000 nm.

When the body fluid is whole blood, the diameter of the beads is preferably 150 to 1,500 μm, such as 200 to 1,000 μm, such as 220 to 750 μm. such as 240 to 500 μm, most preferred 250 to 350 μm.

When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm.

The beads of the fourth matrix 8 may have a porous structure. The pore size preferably ranges from 10 nm to 1,000 nm.

In some examples, the pore size is preferably 10 to 300 nm, such as 50 to 200 nm, such as 75 to 150 nm, such as 100 nm.

In some examples, the pore size is preferably 300 nm to 1,000 nm, such as 400 to 750 nm, such as 450 to 600 nm, such as 500 nm.

Further Embodiments of the Device

According to one embodiment, a sheet or disc of the first matrix 5 or of the third matrix 7 is located at the proximal end A of the device 1.

According to another embodiment, the sheets or discs of the first matrix 5 and/or third matrix 7 are separated from each other by at least one layer comprising the beads of the second matrix 6 and/or fourth matrix 8.

According to yet another embodiment, the device 1 further comprises: at least one sheet or disc of a fifth matrix 9 for adsorption of hydrophobic, hydrophilic or ionic components from a body fluid, wherein the fifth matrix 9 has a porous structure; and/or beads of a sixth matrix 10 for adsorption of hydrophobic, hydrophilic or ionic components from a body fluid.

Proteins have properties relating to charge and degree of hydrophilicity and can be separated from its biological surroundings by adsorption using ionic and/or hydrophobicity structures. Often a combination of the latter is preferred for optimal adsorption.

Examples of such hydrophobic, hydrophilic or ionic components include IgG, albumin and lactate.

Hydrophobic proteins, such as bilirubin, may be bound by a hydrophobic matrix.

Hydrophilic proteins, such as albumin, may be bound by a hydrophilic matrix.

Ionic components, such as bile acids and DNA, may be bound by an ion exchange matrix.

Electronegative surfaces may bind HMGB1, HBP and LBP.

The Fifth Matrix

The fifth matrix 9 has a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The fifth matrix 9 may have an active surface as described above for the first matrix 5.

The fifth matrix 9 may be chosen from the group consisting of a foamed polymer, a molded polymer, a sintered polymer, a polymer cryogel, a nonwoven fabric, and a molecular imprinted polymer that binds the first component.

Preferably, the fifth matrix 9 is a sintered polymer. The polymer may be as described above for the first matrix 5.

Preferably, the fifth matrix 9 is sintered polyethylene. The sintered polyethylene may be functionalized with amino-groups as detailed above.

The fifth matrix 9 may be in the form of a sheet, having a thickness of 1 to 10 mm, such as 2 to 8 mm, such as 4 to 6 mm, preferably about 5 mm.

Preferably, the fifth matrix 9 is in the form of a disc, having a thickness of 1 to mm, such as 2 to 8 mm, such as 4 to 6 mm, preferably about 5 mm. The diameter of such a disc may be 2 to 15 cm, such as 3 to 10 cm, such as 4 to 8 cm, preferably around cm.

The Sixth Matrix

The beads of the sixth matrix 10 may be beads of agarose or beads of polyacrylate, polystyrene, polystyrenedivinylbenzene or copolymers thereof.

Preferably, the beads of the sixth matrix 10 have a diameter of 20 to 1,500 μm and/or a pore size of 10 to 1,000 nm.

When the body fluid is whole blood, the diameter of the beads is preferably 150 to 1,500 μm, such as 200 to 1,000 μm, such as 220 to 750 μm. such as 240 to 500 μm, most preferred 250 to 350 μm.

When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm.

The beads of the sixth matrix 10 may have a porous structure. The pore size preferably ranges from 10 nm to 1,000 nm.

When the hydrophobic, hydrophilic or ionic components are small molecules, such as IgG, the pore size is preferably 10 to 300 nm, such as 50 to 200 nm, such as 75 to 150 nm, such as 100 nm.

When the hydrophobic, hydrophilic or ionic components are large molecules, such as IgM, the pore size is preferably 300 nm to 1,000 nm, such as 400 to 750 nm, such as 450 to 600 nm, such as 500 nm.

The discs and layers of beads may be arranged symmetrically along an axis of the device. In such cases, the direction of flow of the body fluid is not relevant, since the body fluid will encounter the different disc and layers of beads in the same sequence irrespective from which end of the device 1 the body fluid is introduced. A device 1 having such an arrangement of the discs and layers of beads will have the advantage of easy handling, since it can be coupled to the flow of body fluid in two ways.

Method

The method disclosed herein is for binding and separation of at least one component from a body fluid.

General features applicable to the method disclosed herein as well as specific examples of such methods are described below.

A method according to the present disclosure comprises the step of: introducing a body fluid through the inlet 3 of the device 1 according to the present disclosure; passing the body fluid through the first matrix 5 and the second matrix 6, and, if present, through further present matrices 7, 8, 9, 10, of the device 1 according to the present disclosure, whereby said at least one component binds to at least one of the matrices; and collecting the body fluid from the outlet 4 of the device 1 according to the present disclosure.

Use

The present disclosure also relates to the use of a device 1 according to the present disclosure for binding and separation of at least one component from a body fluid.

A device 1 according to the present disclosure may be used in the treatment of a patient suffering from sepsis caused by Gram-negative bacteria. In such cases, the first matrix 5 comprises an LPS-binding peptide. Preferably, the LPS-binding peptide is 4 to amino acids long, more preferably 10 to 35 amino acids long, and in some cases 20 to 30 amino acids long. More preferably, the peptide is a peptide according to SEQ ID NO 1 or a peptide having at least 80%, 85%, 90%, 95% or 99% homology with the peptide according to SEQ ID NO 1. Alternatively, the LPS-binding peptide is a peptide according to SEQ ID NO 2 or a peptide having at least 80%, 85%, 90%, 95% or 99% homology with the peptide according to SEQ ID NO 2. Alternatively, the LPS-binding peptide is a peptide according to SEQ ID NO 3 or a peptide having at least 80%, 85%, 90%, 95% or 99% homology with the peptide according to SEQ ID NO 3. The second matrix 6 may preferably bind a pro-inflammatory cytokine, such as IL-1, IL-6, IL-8, IL-12, IL-17, IL-18, IFN-γ or TNF-α. The device may include further matrices for binding and separating other components, such as further pro-inflammatory cytokines or a complement factor.

A device 1 according to the present disclosure may be used in the treatment of a patient suffering from sepsis caused by Gram-positive bacteria. In such cases, the first matrix 5 comprises an LTA-binding peptide. Preferably, the LTA-binding peptide is 4 to amino acids long, more preferably 6 to 20 amino acids long, and most preferably 8 to 12 amino acids long. More preferably, the peptide is a peptide according to SEQ ID NO 2 or a peptide having at least 80%, 85%, 90%, 95% or 99% homology with the peptide according to SEQ ID NO 2. Alternatively, the LTA-binding peptide is a peptide according to SEQ ID NO 3 or a peptide having at least 80%, 85%, 90%, 95% or 99% homology with the peptide according to SEQ ID NO 3. The second matrix 6 may preferably bind a pro-inflammatory cytokine, such as IL-1, IL-6, IL-8, IL-12, IL-17, IL-18, IFN-γ or TNF-α. The device may include further matrices for binding and separating other components, such as further pro-inflammatory cytokines or a complement factor.

A device 1 according to the present disclosure may be used in the treatment of a patient suffering from a disease associated with autoantibodies. In such cases, the first matrix 5 comprises an antibody-binding moiety. The second matrix 6 may preferably bind a pro-inflammatory cytokine, such as IL-1, IL-6, IL-8, IL-12, IL-17, IL-18, IFN-γ or TNF-α. The device may include further matrices for binding and separating other components, such as further pro-inflammatory cytokines or a complement factor.

In connection with surgical procedures, severe burns and other trauma, endotoxins, such as LPS and LTA, from bacteria in the guts may translocate into the blood stream, causing a sepsis-like condition.

Thus, a device 1 according to the present disclosure may be used in the treatment of a patient suffering from a sepsis-like condition after a surgical procedure. In such cases, the first matrix 5 comprises an LPS-binding peptide and/or an LTA-binding peptide. Preferably, the LPS-binding peptide is 4 to 40 amino acids long, more preferably 10 to 35 amino acids long, and in some cases 20 to 30 amino acids long. More preferably, the peptide is a peptide according to SEQ ID NO 1 or a peptide having at least 80%, 85%, 90%, 95% or 99% homology with the peptide according to SEQ ID NO 1. Alternatively, the LPS/LTA-binding peptide is a peptide according to SEQ ID NO 2 or a peptide having at least 80%, 85%, 90%, 95% or 99% homology with the peptide according to SEQ ID NO 2. Alternatively, the LPS/LTA-binding peptide is a peptide according to SEQ ID NO 3 or a peptide having at least 80%, 85%, 90%, 95% or 99% homology with the peptide according to SEQ ID NO 3. The second matrix 6 may preferably bind a pro-inflammatory cytokine, such as IL-1, IL-6, IL-8, IL-12, IL-17, IL-18, IFN-γ or TNF-α. The device may include further matrices for binding and separating other components, such as further pro-inflammatory cytokines or a complement factor.

Thus, a device 1 according to the present disclosure may be used in the treatment of a patient suffering from a sepsis-like condition after severe burns. In such cases, the first matrix 5 comprises an LPS-binding peptide and/or an LTA-binding peptide. Preferably, the LPS-binding peptide is 4 to 40 amino acids long, more preferably 10 to amino acids long, and in some cases 20 to 30 amino acids long. More preferably, the peptide is a peptide according to SEQ ID NO 1 or a peptide having at least 80%, 85%, 90%, 95% or 99% homology with the peptide according to SEQ ID NO 1. Alternatively, the LPS/LTA-binding peptide is a peptide according to SEQ ID NO 2 or a peptide having at least 80%, 85%, 90%, 95% or 99% homology with the peptide according to SEQ ID NO 2. Alternatively, the LPS/LTA-binding peptide is a peptide according to SEQ ID NO 3 or a peptide having at least 80%, 85%, 90%, 95% or 99% homology with the peptide according to SEQ ID NO 3. The second matrix 6 may preferably bind a pro-inflammatory cytokine, such as IL-1, IL-6, IL-8, IL-12, IL-17, IL-18, IFN-γ or TNF-α. The device may include further matrices for binding and separating other components, such as further pro-inflammatory cytokines or a complement factor.

Thus, a device 1 according to the present disclosure may be used in the treatment of a patient suffering from a sepsis-like condition after trauma. In such cases, the first matrix 5 comprises an LPS-binding peptide and/or an LTA-binding peptide. Preferably, the LPS-binding peptide is 4 to 40 amino acids long, more preferably 10 to 35 amino acids long, and in some cases 20 to 30 amino acids long. More preferably, the peptide is a peptide according to SEQ ID NO 1 or a peptide having at least 80%, 85%, 90%, 95% or 99% homology with the peptide according to SEQ ID NO 1. Alternatively, the LPS/LTA-binding peptide is a peptide according to SEQ ID NO 2 or a peptide having at least 80%, 85%, 90%, 95% or 99% homology with the peptide according to SEQ ID NO 2. Alternatively, the LPS/LTA-binding peptide is a peptide according to SEQ ID NO 3 or a peptide having at least 80%, 85%, 90%, 95% or 99% homology with the peptide according to SEQ ID NO 3. The second matrix 6 may preferably bind a pro-inflammatory cytokine, such as IL-1, IL-6, IL-8, IL-12, IL-17, IL-18, IFN-γ or TNF-α. The device may include further matrices for binding and separating other components, such as further pro-inflammatory cytokines or a complement factor.

### SPECIFIC EMBODIMENTS

In the following, with reference to the figures, examples of devices according to the present disclosure will be described. Effects and advantages of specific features are, unless stated otherwise, as described above in the general section.

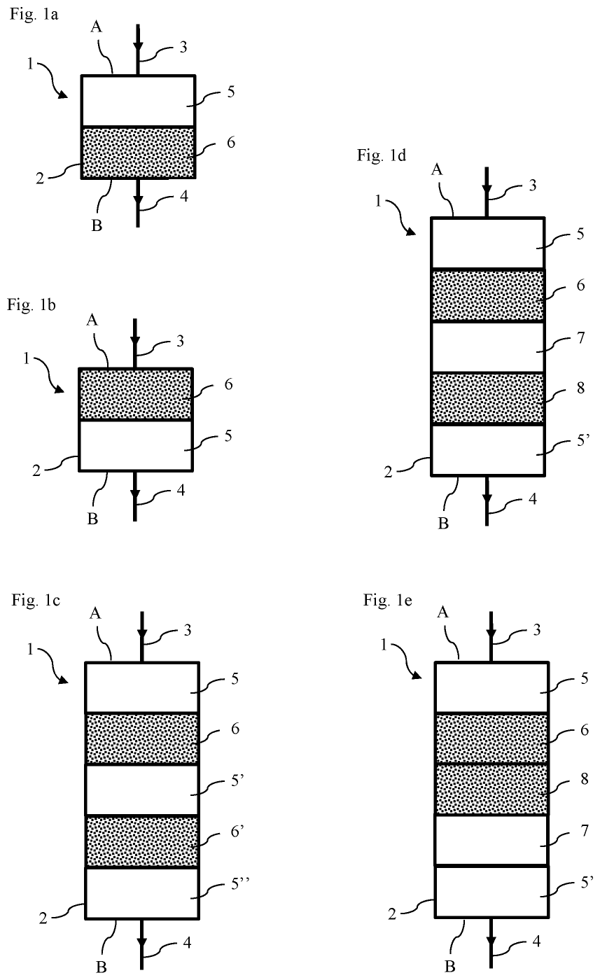

FIGS. 1a-1e show different embodiments of a device according to the present disclosure. The devices 1 shown in FIGS. 1a-1e have a housing 2, an inlet 3 located at the proximal end A of the device 1 and an outlet 4 located at the distal end B of the device. The device comprises a sheet or disc of the first matrix 5 and beads of the second matrix 6 (FIGS. 1a-1b). The device may further comprise a further sheet or disc of the first matrix 5′, and a further layer of beads of the second matrix 6′ (FIG. 1c). The device may further comprise a sheet or disc of the third matrix 7, and beads of the fourth matrix 8 (FIGS. 1d-1e).

The nature of the matrices is as described above in the general description.

The sheets or discs of the first matrix 5 and the third matrix 7 prevent channeling and distributes the body fluid over the area of the discs, thus creating a substantially uniform distribution of the body fluid. Thus, the sheets or discs of the first matrix 5 and of the third matrix 7 distribute the flow of the body fluid, such that more of the available adsorption capacity of the beads of the second matrix 6 or of the fourth matrix 8 can be utilized. In addition, when passing through the sheets or discs of the first matrix 5 and the third matrix 7, the body fluid is mixed. This leads to even concentrations of the components on the body fluid. Thus, concentration gradients are avoided and an increased part of the body fluid is in contact with the matrices.

The beads of the second matrix 6 and of the fourth matrix 8 provide a large surface are where a component of the body fluid can bind.

In its simplest form, a device according to the present disclosure contains one sheet or disc of the first matrix 5 and one layer of beads of the second matrix 6 (FIGS. 1a and 1b). Preferably, the matrices are arranged such that the body fluid first passes the first matrix 5, where it is mixed and distributed over the area of the interior of the device 1 before it encounters the beads of the second matrix 6 (FIG. 1a). However, in certain embodiments, the body fluid first passes the beads of the second matrix 6 and then the sheet or disc of the first matrix 5 (FIG. 1b).

The device in FIG. 1c comprises, from the proximal end A to the distal end B, a disc of the first matrix 5, a layer of beads of the second matrix 6, a disc of the first matrix 5′, a layer of beads of the second matrix 6′, and a disc of the first matrix 5″.

The device in FIG. 1d comprises, from the proximal end A to the distal end B, a disc of the first matrix 5, a layer of beads of the second matrix 6, a disc of the third matrix 7, a layer of beads of the fourth matrix 8, and a disc of the first matrix 5′.

The device in FIG. 1e comprises, from the proximal end A to the distal end B, a disc of the first matrix 5, a layer of beads of the second matrix 6, a layer of beads of the fourth matrix 8, a disc of the third matrix 7, and a disc of the first matrix 5′.

The skilled person realizes that the discs and beads can be arranged in different orders and that the discs and beads can bind the same or different components of a body fluid.

Especially, the discs and layers of beads may be arranged symmetrically along an axis of the device. In such cases, the direction of flow of the body fluid is not relevant, since the body fluid will encounter the different disc and layers of beads in the same sequence irrespective from which end of the device 1 the body fluid is introduced. A device 1 having such an arrangement of the discs and layers of beads will have the advantage of easy handling, since it can be coupled to the flow of body fluid in two ways.

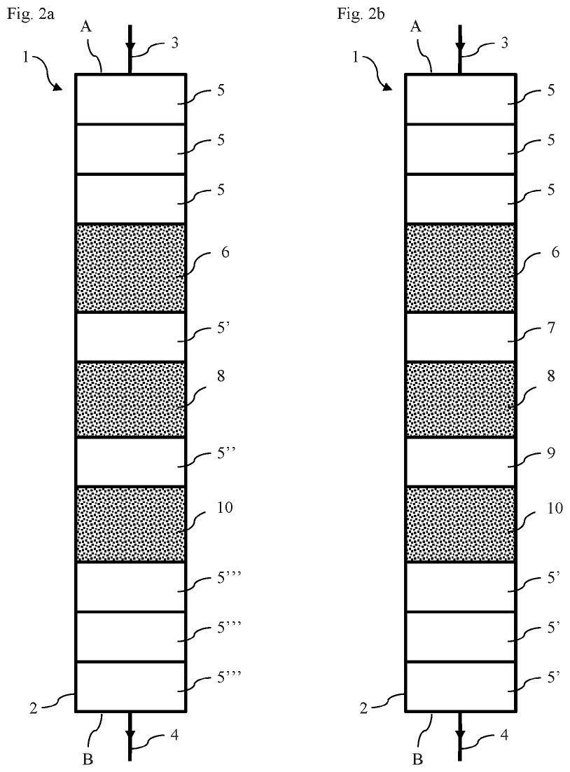

FIG. 2a shows a preferred embodiment comprising different matrices. The device 1 shown in FIG. 2a has a housing 2, an inlet 3 located at the proximal end A of the device 1 and an outlet 4 located at the distal end B of the device. The device comprises, from the proximal end A to the distal end B, three discs of the first matrix 5, a layer of beads of the second matrix 6, a disc of the first matrix 5′, a layer of beads of the fourth matrix 8, a disc of the first matrix 5″, a layer of beads of the sixth matrix 10, and three discs of the first matrix 5′″.

The first three discs of the first matrix 5 mix the body fluid, prevent channeling and distribute the body fluid over the area of the discs, thus creating a substantially uniform distribution of the body fluid. In this particular device, the first matrix 5 binds LPS via an LPS-binding peptide bound to the first matrix 5. Preferably, the peptide is a peptide according to SEQ ID 1.

After the first thee discs of the first matrix 5, from the proximal end A to the distal end B, a layer of beads of a second matrix 6 is located. The beads provide a large surface are where a component of the body fluid can bind. The beads adsorb LPS. This may be accomplished by an LPS-binding peptide covalently bound to the beads.

After the layer of beads of the second matrix 6, from the proximal end A to the distal end B, a further disc of the first matrix 5′ is located. This disc mixes the body fluid, prevents channeling and distributes the body fluid over the area of the disc, thus creating a substantially uniform distribution of the body fluid. In this particular device, the first matrix 5′ binds LPS via an LPS-binding peptide bound to the first matrix 5′. Preferably, the peptide is a peptide according to SEQ ID 1.

After the disc of the first matrix 5′, from the proximal end A to the distal end B, a layer of beads of a fourth matrix 8 is located. The beads provide a large surface are where a component of the body fluid can bind. This layer of beads adsorbs a pro-inflammatory cytokine, preferably IL-1, IL-6, IL-8, IL-12, IL-17, IL-18, IFN-γ or TNF-α.

After the layer of beads of the fourth matrix 8, from the proximal end A to the distal end B, a further disc of the first matrix 5″ is located. The disc of the first matrix mixes the body fluid, prevents channeling and distributes the body fluid over the area of the disc, thus creating a substantially uniform distribution of the body fluid. In this particular device, the first matrix 5″ binds LPS via an LPS-binding peptide bound to the first matrix 5″. Preferably, the peptide is a peptide according to SEQ ID 1.

After the disc of the first matrix 5″, from the proximal end A to the distal end B, a layer of beads of a sixth matrix 10 is located. The beads provide a large surface are where components of the body fluid can bind.

After the layer of beads of the sixth matrix 10, from the proximal end A to the distal end B, three further discs of the first matrix 5′″ are located. In this particular device, the first matrix 5 binds LPS via an LPS-binding peptide bound to the first matrix Preferably, the peptide is a peptide according to SEQ ID 1.

Preferably, the discs of the first matrix 5, 5′, 5″, 5′″ have a thickness of 1 to 10 mm, such as 2 to 8 mm, such as 4 to 6 mm, preferably about 5 mm. The diameter of such a disc is preferably 2 to 15 cm, such as 3 to 10 cm, such as 4 to 8 cm, preferably around 5 cm.

The first matrix 5, 5′, 5″, 5′″ has a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The beads of the second matrix 6 preferably have a diameter of 20 to 1,500 μm and/or a pore size of 10 to 1,000 nm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm. The beads of the second matrix 6 may have a porous structure. The pore size preferably ranges from 10 to 300 nm, such as 50 to 200 nm, such as 75 to 150 nm, such as 100 nm.

The beads of the fourth matrix 8 preferably have a diameter of 20 to 1,500 μm and/or a pore size of 10 to 1,000 nm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm. The beads of the fourth matrix 8 may have a porous structure. The pore size preferably ranges from 10 to 300 nm, such as 50 to 200 nm, such as 75 to 150 nm, such as 100 nm.

The beads of the sixth matrix 10 preferably have a diameter of 20 to 1,500 μm and/or a pore size of 10 to 1,000 nm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm. The beads of the sixth matrix 10 may have a porous structure. The pore size preferably ranges from 10 to 300 nm, such as 50 to 200 nm, such as 75 to 150 nm, such as 100 nm.

The device 1 of FIG. 2a may be used in the treatment of sepsis caused by gram-negative bacteria.

The device 1 of FIG. 2a is an example of a symmetrical device 1. As explained above, in such a device 1, the discs and layers of beads are arranged symmetrically along the axis of the device. In such cases, the direction of flow of the body fluid is not relevant, since the body fluid will encounter the different disc and layers of beads in the same sequence irrespective from which end of the device 1 the body fluid is introduced. A device 1 having such an arrangement of the discs and layers of beads will have the advantage of easy handling, since it can be coupled to the flow of body fluid in two ways.

An advantage of the combination of the discs and beads is that the discs, in addition to binding LPS, mix the body fluid and distribute the body fluid over substantially the entire area of the cross section of the device, thus making it possible to utilize more of the adsorption capacity of the volume of beads. Thus, less beads may be used to achieve the same adsorption capacity of a device not comprising the discs.

Another advantage of this device is that both the cause (LPS) of the inflammatory response as well as the endogenous mediators of the inflammations (pro-inflammatory cytokines) are bound by the matrices of the device 1 and are separated from the blood or plasma which is passed through the device 1.

Furthermore, the beads of the sixth matrix 10 adsorb different types of inflammatory mediators present in excess in the body fluid. This may mitigate multiorgan dysfunction or failure.

FIG. 2b shows another embodiment comprising different matrices. The device 1 shown in FIG. 2b has a housing 2, an inlet 3 located at the proximal end A of the device 1 and an outlet 4 located at the distal end B of the device. The device comprises, from the proximal end to the distal end, three discs of the first matrix 5, a layer of beads of the second matrix 6, a disc of the third matrix 7, a layer of beads of the fourth matrix 8, a disc of the fifth matrix 9, a layer of beads of the sixth matrix 10, and three discs of the first matrix 5′.

The first three discs of the first matrix 5 mix the body fluid, prevent channeling and distribute the body fluid over the area of the discs, thus creating a substantially uniform distribution of the body fluid. In this particular device, the first matrix 5 binds LPS via an LPS-binding peptide bound to the first matrix 5. Preferably, the peptide is a peptide according to SEQ ID 1.

After the first thee discs of the first matrix 5, from the proximal end A to the distal end B, a layer of beads of a second matrix 6 is located. The beads provide a large surface are where a component of the body fluid can bind. This layer adsorbs LPS. This may be accomplished by an LPS-binding peptide covalently bound to the beads.

After the layer of beads of the second matrix 6, from the proximal end A to the distal end B, a disc of the third matrix 7 is located. This disc mixes the body fluid, prevents channeling and distributes the body fluid over the area of the disc, thus creating a substantially uniform distribution of the body fluid. In this particular device, the third matrix 7 binds a pro-inflammatory cytokine, preferably IL-1, IL-6, IL-8, IL-12, IL-17, IL-18, IFN-γ or TNF-α.

After the disc of the third matrix 7, from the proximal end A to the distal end B, a layer of beads of a fourth matrix 8 is located. The beads provide a large surface are where a component of the body fluid can bind. These beads adsorb a pro-inflammatory cytokine, preferably IL-1, IL-6, IL-8, IL-12, IL-17, IL-18, IFN-γ or TNF-α.

After the layer of beads of the fourth matrix 8, from the proximal end A to the distal end B, a disc of the fifth matrix 9 is located. This disc mixes the body fluid, prevents channeling and distributes the body fluid over the area of the disc, thus creating a substantially uniform distribution of the body fluid. In this particular device, the fifth matrix 9 adsorb different types of inflammatory mediators present in excess in the body fluid. This may mitigate multiorgan dysfunction or failure.

After the disc of the fifth matrix 9, from the proximal end A to the distal end B, a layer of beads of a sixth matrix 10 is located. The beads provide a large surface are where a component of the body fluid can bind. These beads adsorb different types of inflammatory mediators present in excess in the body fluid. This may mitigate multiorgan dysfunction or failure.

After the layer of beads of the sixth matrix 10, from the proximal end A to the distal end B, a three further discs of the first matrix 5′″ are located. In this particular device, the first matrix 5 binds LPS via an LPS-binding peptide bound to the first matrix Preferably, the peptide is a peptide according to SEQ ID 1.

Preferably, the discs of the first matrix 5, 5′, the third matrix 7, and the fifth matrix 9 have a thickness of 1 to 10 mm, such as 2 to 8 mm, such as 4 to 6 mm, preferably about 5 mm. The diameter of such a disc is preferably 2 to 15 cm, such as 3 to 10 cm, such as 4 to 8 cm, preferably around 5 cm.

The first matrix 5, 5′, the third matrix 7, and the fifth matrix 9 have a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The beads of the second matrix 6 preferably have a diameter of 20 to 1,500 μm and/or a pore size of 10 to 1,000 nm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm. The beads of the second matrix 6 may have a porous structure. The pore size preferably ranges from 10 to 300 nm, such as 50 to 200 nm, such as 75 to 150 nm, such as 100 nm.

The beads of the fourth matrix 8 preferably have a diameter of 20 to 1,500 μm and/or a pore size of 10 to 1,000 nm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm. The beads of the fourth matrix 8 may have a porous structure. The pore size preferably ranges from 10 to 300 nm, such as 50 to 200 nm, such as 75 to 150 nm, such as 100 nm.

The beads of the sixth matrix 10 preferably have a diameter of 20 to 1,500 μm and/or a pore size of 10 to 1,000 nm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm. The beads of the sixth matrix 10 may have a porous structure. The pore size preferably ranges from 10 to 300 nm, such as 50 to 200 nm, such as 75 to 150 nm, such as 100 nm.

The device 1 of FIG. 2b may be used in the treatment of sepsis caused by gram-negative bacteria.

An advantage of the combination of the discs and beads is that the discs, in addition to binding LPS, mix the body fluid and distribute the body fluid over substantially the entire area of the cross section of the device, thus making it possible to utilize more of the adsorption capacity of the volume of beads. Thus, less beads may be used to achieve the same adsorption capacity of a device not comprising the discs.

Another advantage of this device is that both the cause (LPS) of the inflammatory response as well as the endogenous mediators of the inflammations (pro-inflammatory cytokines) are bound by the matrices of the device 1 and are separated from the blood or plasma which is passed through the device 1.

Furthermore, the beads of the sixth matrix 10 adsorb different types of inflammatory mediators present in excess in the body fluid. This may mitigate multiorgan dysfunction or failure.

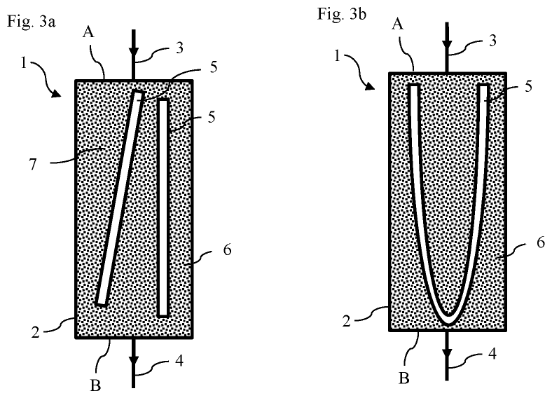

FIG. 3a shows another embodiment of a device according to the present disclosure. The device 1 shown in FIG. 3a has a housing 2, an inlet 3 located at the proximal end A of the device 1 and an outlet 4 located at the distal end B of the device. The device comprises sheets of the first matrix 5 surrounded by beads of the second matrix 6.

Preferably, the sheets of the first matrix 5 comprise an LPS-binding peptide, such as an LPS-binding peptide according to SEQ ID NO 1 or a peptide having at least 80%, 85%, 90%, 95% or 99% homology with the LPS-binding peptide according to SEQ ID NO 1; and/or an LPS/LTA-binding peptide, such as an LPS/LTA-binding peptide according to SEQ ID NO 2 or a peptide having at least 80%, 85%, 90%, 95% or 99% homology with the LPS-binding peptide according to SEQ ID NO 2; or such as an LPS/LTA-binding peptide according to SEQ ID NO 3 or a peptide having at least 80%, 85%, 90%, 95% or 99% homology with the LPS/LTA-binding peptide according to SEQ ID NO 3. Thus, the device may be used in the treatment of sepsis caused by Gram-negative and Gram-positive bacteria.

Preferably, the sheets of the first matrix 5 have a thickness of 1 to 10 mm, such as 2 to 8 mm, such as 4 to 6 mm, preferably about 5 mm.

The first matrix 5 has a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The beads of the second matrix 6 may comprise an LPS-binding peptide, such as an LPS-binding peptide according to SEQ ID NO 1 or a peptide having at least 80%, 85%, 90%, 95% or 99% homology with the LPS-binding peptide according to SEQ ID NO 1; and/or an LPS/LTA-binding peptide, such as an LPS/LTA-binding peptide according to SEQ ID NO 2 or a peptide having at least 80%, 85%, 90%, 95% or 99% homology with the LPS/LTA-binding peptide according to SEQ ID NO 2; or an LPS/LTA-binding peptide according to SEQ ID NO 3 or a peptide having at least 80%, 85%, 90%, 95% or 99% homology with the LPS/LTA-binding peptide according to SEQ ID NO 3.

Alternatively, the beads of the second matrix 6 may comprise a moiety that binds a cytokine, preferably a pro-inflammatory cytokine, such as IL-1, IL-6, IL-8, IL-12, IL-17, IL-18, IFN-γ or TNF-α. Preferably, the moiety is an antibody or an antibody fragment.

The beads of the second matrix 6 provide a large surface are where a component of the body fluid can bind.

The beads of the second matrix 6 have a diameter of 20 to 1,500 μm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm.

The beads of the second matrix 6 may have a porous structure. The pore size preferably ranges from 10 nm to 1,000 nm. When the second component is a small molecule, such as IgG, the pore size is preferably 10 to 300 nm, such as 50 to 200 nm, such as 75 to 150 nm, such as 100 nm. When the second component is a large molecule, such as IgM, the pore size is preferably 300 nm to 1,000 nm, such as 400 to 750 nm, such as 450 to 600 nm, such as 500 nm.

FIG. 3b shows another embodiment of a device according to the present disclosure. The device 1 shown in FIG. 3a has a housing 2, an inlet 3 located at the proximal end A of the device 1 and an outlet 4 located at the distal end B of the device. The device comprises a sheet of the first matrix 5 formed as a tube with one closed end surrounded by beads of the second matrix 6. Thus, in this embodiment, the sheet is formed into a three-dimensional body.

Preferably, the sheet of the first matrix 5 comprises an LPS-binding peptide, such as an LPS-binding peptide according to SEQ ID NO 1 or a peptide having at least 80%, 85%, 90%, 95% or 99% homology with the LPS-binding peptide according to SEQ ID NO 1; and/or an LPS/LTA-binding peptide, such as an LPS/LTA-binding peptide according to SEQ ID NO 2 or a peptide having at least 80%, 85%, 90%, 95% or 99% homology with the LPS/LTA-binding peptide according to SEQ ID NO 2; or an LPS/LTA-binding peptide according to SEQ ID NO 3 or a peptide having at least 80%, 85%, 90%, 95% or 99% homology with the LPS/LTA-binding peptide according to SEQ ID NO 3. Thus, the device may be used in the treatment of sepsis caused by Gram-negative and Gram-positive bacteria.

Preferably, the sheet of the first matrix 5 has a thickness of 1 to 10 mm, such as 2 to 8 mm, such as 4 to 6 mm, preferably about 5 mm.

The first matrix 5 has a porous structure. The pore size preferably ranges from 1 μm to 500 μm in diameter, more preferably from 70 μm to 170 μm, most preferred from 80 μm to 100 μm. Such pore sizes enable that high flow rates of whole blood may be maintained without cellular damage or cellular exclusion. When the body fluid does not contain any blood cells, the pore size may be 1 μm to 25 μm.

The sheet of the first matrix 5 distributes the flow of the body fluid, such that more of the available adsorption capacity of the beads of the second matrix 6 can be utilized. Further, the sheet of the first matrix 5 mixes the body fluid.

The beads of the second matrix 6 may comprise an LPS-binding peptide, such as an LPS-binding peptide according to SEQ ID NO 1 or a peptide having at least 80%, 85%, 90%, 95% or 99% homology with the LPS-binding peptide according to SEQ ID NO 1; and/or an LPS/LTA-binding peptide, such as an LPS/LTA-binding peptide according to SEQ ID NO 2 or a peptide having at least 80%, 85%, 90%, 95% or 99% homology with the LPS/LTA-binding peptide according to SEQ ID NO 2; or an LPS/LTA-binding peptide according to SEQ ID NO 3 or a peptide having at least 80%, 85%, 90%, 95% or 99% homology with the LPS/LTA-binding peptide according to SEQ ID NO 3.

Alternatively, the beads of the second matrix 6 may comprise a moiety that binds a cytokine, preferably a pro-inflammatory cytokine, such as IL-1, IL-6, IL-8, IL-12, IL-17, IL-18, IFN-γ or TNF-α. Preferably, the moiety is an antibody or an antibody fragment.

The beads of the second matrix 6 provide a large surface are where a component of the body fluid can bind.

The beads of the second matrix 6 have a diameter of 20 to 1,500 μm. When the body fluid is whole blood, the diameter of the beads is preferably 150 to 1,500 μm, such as 200 to 1,000 μm, such as 220 to 750 μm. such as 240 to 500 μm, most preferred 250 to 350 μm. When the body fluid does not contain any blood cells, e.g. when the body fluid is plasma, the diameter of the beads is preferably 20 to 150 μm, such as 50 to 130 μm, such as 60 to 115 μm, such as 70 to 100 μm, preferably 80 to 90 μm.

The beads of the second matrix 6 may have a porous structure. The pore size preferably ranges from 10 nm to 1,000 nm. In some examples, the pore size is preferably to 300 nm, such as 50 to 200 nm, such as 75 to 150 nm, such as 100 nm. In some examples, the pore size is preferably 300 nm to 1,000 nm, such as 400 to 750 nm, such as 450 to 600 nm, such as 500 nm.

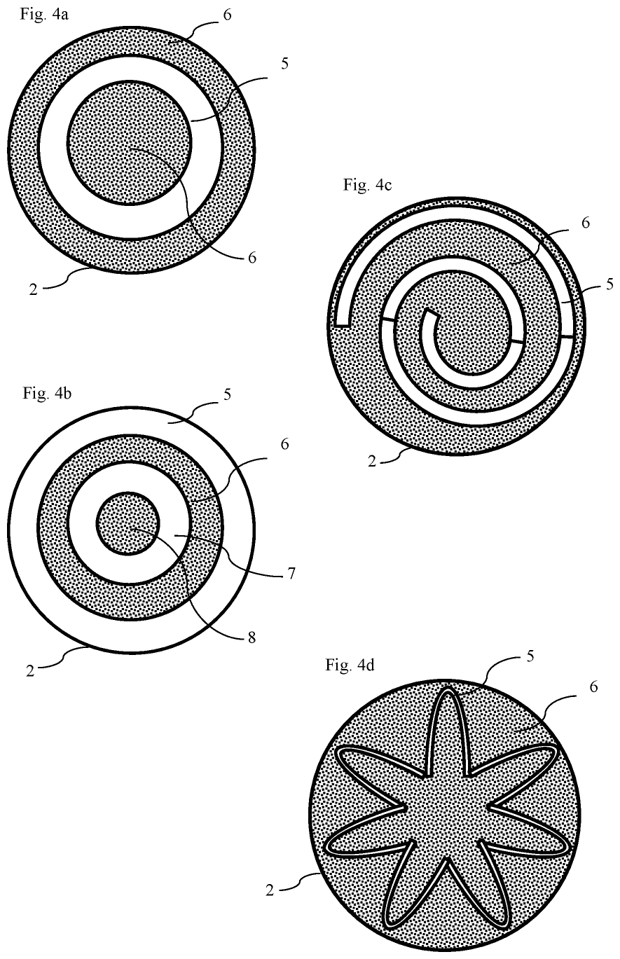

FIGS. 4a-4d show cross sections of different embodiments of a device 1 according to the present disclosure. The nature of the matrices is as described above in the general description. The sheets of the first matrix 5 distribute the flow of the body fluid, such that more of the available adsorption capacity of the beads of the second matrix 6 can be utilized. Further, the sheets of the first matrix 5 mix the body fluid. The beads of the second matrix 6 provide a large surface are where a component of the body fluid can bind.

In FIG. 4a, a sheet of the first matrix 5 is formed as a tube surrounded by beads of the second matrix 6.

In FIG. 4b, the device comprises a sheet of the first matrix 5 is formed as a tube and a sheet of the third matrix 7 is formed as a tub. Between these two tubes, beads of the second matrix 6 are located and inside the tube of the third matrix 7, beads of the fourth matrix 8 are located.

The device shown in FIG. 4c resembles a “roll cake” wherein a sheet of the first matrix 5 is rolled around beads of the second matrix 6. In other words, the cross section shows the sheet of the first matrix 5 rolled in a loose spiral form and surrounded by beads of the second matrix 6.

In the device shown in FIG. 4d, a plurality of bent sheets of the first matrix 5 are surrounded by beads of the second matrix 6. The skilled person realizes that, in other embodiments, sheets of different matrices may be used.

Amino Acid Sequence

SEQ ID NO 1:
HAEHKVKIKVKQKYGQFPQGTEVTYTC

SEQ ID NO 1 originates from horseshoe crab (Limulus polyphemus).

SEQ ID NO 2:
QRLFQVKGRR

SEQ ID NO 2 is a modified human gelsolin, an actin-binding protein

SEQ ID NO 3:
RRWVRRVRRWVRRVVRVVRRWVRR

SEQ ID NO 3 is a modified lentivirus lytic peptide. Lentivirus is a genus of retroviruses.

1. A device for binding and separation of at least one component from a body fluid, the device comprising:
a proximal end;
a distal end;
a housing;
an inlet disposed at the proximal end;
an outlet;
at least one of a sheet and a disc of a first matrix for binding of a first component from the body fluid, the first matrix having a porous structure; and
a plurality of beads of a second matrix for binding of a second component from the body fluid;
wherein the first component and the second component are one of the same and different.

2. The device according to claim 1, wherein at least one of the first component and the second component is at least one of:
an endotoxin;
a cytokine;
a complement factor;
histone;
an autoantibody;
macrophage migration inhibitory factor (MIF);
high mobility group protein 1 (HMGB1);
heparin-binding protein (HBP);
lipopolysaccharide-binding protein (LBP);
DNA; and
fragments thereof.

3. The device according to claim 1, wherein:
the first component is one of LPS and LTA; and
the second component is one of a pro-inflammatory cytokine and an anti-inflammatory cytokine.

4. The device according to claim 1, wherein the first matrix includes a moiety that specifically binds the first component.

5. The device according to claim 1, wherein the first matrix includes an endotoxin-binding peptide, and wherein the endotoxin-binding peptide is one of:
an LPS-binding peptide according to SEQ ID NO 1;
a peptide having at least 80% homology with an LPS-binding peptide according to SEQ ID NO 1;
an LPS/LTA-binding peptide according to SEQ ID NO 2;
a peptide having at least 80% homology with an LPS/LTA-binding peptide according to SEQ ID NO 2;
an LPS/LTA-binding peptide according to SEQ ID NO 3; and
a peptide having at least 80% homology with an LPS/LTA-binding peptide according to SEQ ID NO 3.

6. The device according to claim 1, wherein the first matrix is at least one of a foamed polymer, a molded polymer, a sintered polymer, a polymer cryogel, a nonwoven fabric, and a molecular imprinted polymer that binds the first component.

7. The device according to claim 1, wherein the first matrix is sintered polyethylene.

8. The device according to claim 1, wherein the second matrix includes a moiety that specifically binds the second component.

9. The device according to claim 1, wherein the plurality of beads of the second matrix are beads of one of agarose, polyacrylate, polystyrene, polystyrenedivinylbenzene, and copolymers thereof.

10. The device according to claim 1, wherein the plurality of beads of the second matrix have at least one of:
a diameter of 20 to 1,500 μm; and
a pore size of 10 to 1,000 nm.

11. The device according to claim 1, wherein the at least one of the sheet and the disc of the first matrix includes at least one of:
a plurality of sheets of the first matrix; and
a plurality of discs of the first matrix.

12. The device according to claim 11, wherein at least one of the plurality of sheets and the plurality of discs of the first matrix are separated from each other via at least one layer including the plurality of beads of the second matrix.

13. The device according to claim 1, further comprising at least one of:
at least one of a sheet and a disc of a third matrix for binding of a third component from the body fluid, the third matrix having a porous structure; and
a plurality of beads of a fourth matrix for binding of a fourth component from the body fluid.

14. The device according to claim 13, wherein at least one of the third component and the fourth component is at least one of:
an endotoxin;
a cytokine;
a complement factor;
histone;
an autoantibody;
macrophage migration inhibitory factor (MIF);
high mobility group protein 1 (HMGB1);
heparin-binding protein (HBP);
lipopolysaccharide-binding protein (LBP);
DNA; and
fragments thereof.

15. The device according to claim 13, wherein at least one of:
the third matrix includes a moiety that specifically binds the third component; and
the fourth matrix includes a moiety that specifically binds the fourth component.

16. The device according to claim 13, wherein at least one of (i) the at least one of the sheet and the disc of the first matrix and (ii) the at least one of the sheet and the disc of the third matrix is disposed at the proximal end.

17. The device according to claim 13, wherein the at least one of the sheet and the disc of the first matrix and the at least one of the sheet and the disc of the third matrix are separated from each other by at least one layer including at least one of the plurality of beads of the second matrix and the plurality of beads of the fourth matrix.

18. The device according to claim 13, further comprising at least one of:
at least one of a sheet and a disc of a fifth matrix for adsorption of at least one of a hydrophobic component, a hydrophilic component, and an ionic component from the body fluid, the fifth matrix having a porous structure; and
a plurality of beads of a sixth matrix for adsorption of at least one of a hydrophobic component, a hydrophilic component, and an ionic component from the body fluid.

19. A method for binding and separating at least one component from a body fluid, comprising:
providing the device according to claim 1;
introducing the body fluid through the inlet of the device;
binding at least one of the first component to the first matrix and the second component to the second matrix via passing the body fluid through the first matrix and the second matrix; and
collecting the body fluid from the outlet of the device.

20. A method of using the device according to claim 1, comprising binding and separating at least one of the first component and the second component from the body fluid via flowing the body fluid through the inlet, the first matrix, the second matrix, and the outlet of the device.
